(19) United States
(12) Patent Application Publication
(10) Pub. No.: US 2024/0000814 A1
(43) Pub. Date: Jan. 4, 2024

(54) TREATING ALCOHOL USE DISORDER USING PSILOCYBIN

(71) Applicant: New York University, New York, NY (US)

(72) Inventor: Michael Bogenschutz, New York, NY (US)

(21) Appl. No.: 18/153,140

(22) Filed: Sep. 22, 2023

Related U.S. Application Data
(60) Provisional application No. 63/373,243, filed on Aug. 23, 2022.
(60) Provisional application No. 63/298,496, filed on Jan. 11, 2022.

(51) Int. Cl. A61K 31/675; A61K 31/5513; A61P 25/32

(52) U.S. Cl. CPC A61K 31/675; A61K 31/5513; A61P 25/32

(57) ABSTRACT

This invention provides methods for treating a human subject afflicted with alcohol use disorder (AUD) comprising administering to the subject a therapeutically effective amount of a psilocybin-based compound.

This application claims the benefit of U.S. Provisional Application No. 63/298,496, filed Jan. 11, 2022, and U.S. Provisional Application No. 63/373,243, filed Aug. 23, 2022, the contents of both of which are incorporated herein by reference.

Throughout this application, various publications are referenced. The disclosures of these publications in their entireties are hereby incorporated by reference into this application in order to more fully describe the state of the art to which this invention pertains.

This invention was made with government support under Grant No. UL1 TR000038 awarded by the National Center for Advancing Translational Sciences, the National Institutes of Health. The government has certain rights in the invention.

### Field of the Invention

The present invention relates to compositions and methods for treating alcohol use disorder. More specifically, the present invention provides methods of treating alcohol use disorder with psilocybin.

### BACKGROUND OF THE INVENTION

Alcohol is ranked among the most harmful drugs of abuse in the United States and globally, and alcohol-related mortality in the U.S. is on the rise. Alcohol use disorder (AUD) is a disease that is generally characterized by compulsive alcohol use, loss of control over alcohol intake, and a negative emotional state when not using. This disease affects about 15 million people in the United States, including adolescents and adults, as well as their families.

Several types of treatment are available for AUD. Behavioral treatments like counseling can help change an individual's drinking behavior, such as providing coping mechanisms and suggesting that the individual avoid triggers that cause drinking. There are also many support groups such as Alcoholics Anonymous that provide peer support to help individuals to stop drinking. Medications can also be prescribed to help stop or reduce drinking.

Naltrexone is a drug that acts as a competitive antagonist at the μ-opioid receptor. This drug is prescribed to manage alcohol or opioid dependence. Naltrexone can decrease the amount and frequency of drinking. However, it seems to only have a modest effect on AUD. In treating AUD, it is taken orally about an hour before drinking to avoid side effects. Naltrexone blocks the positive-reinforcement effects of alcohol. It can decrease cravings for opioids as well after a few weeks and can decrease the risk of overdose. For this indication, naltrexone is injected once a month. There are several side effects, including diarrhea, abdominal cramping, liver damage, trouble sleeping, anxiety, nausea, and headaches.

Acamprosate stabilizes chemical signaling in the brain that would otherwise be disrupted by alcohol withdrawal. It has not been found to be effective alone, and requires psychosocial support. Side effects include allergic reactions, abnormal heart rhythms, low or high blood pressure, diarrhea, headaches, insomnia, and impotence. Major side effects can include suicidal behavior, major depressive disorder, and kidney failure.

Disulfiram produces an acute sensitivity to ethanol by inhibiting the enzyme acetaldehyde dehydrogenase. This effectively produces a hangover effect immediately after drinking. Side effects include flushing, throbbing in the head and neck, headaches, respiratory difficulty, nausea, vomiting, sweating, thirst, chest pain, palpitations, dyspnea, hyperventilation, fast heart rate, low blood pressure, fainting, uneasiness, weakness, vertigo, blurred vision, and confusion. Severe side effects include respiratory depression, cardiovascular collapse, abnormal heart rhythms, heart attack, acute congestive heart failure, unconsciousness, convulsions, and death.

Drugs that are used for other indications can also be helpful in treating AUD, including varenicline (anti-smoking), gabapentin (pain and epilepsy), and topiramate (anti-epileptic).

Psilocybin is a psychedelic that is metabolized in the body to psilocin, which is an agonist for serotonin receptors and binds to 5-HT2A with high affinity and to 5-HT1 with low affinity. Psilocin can indirectly increase concentrations of dopamine in the body, though it has no effect itself on the dopamine receptor.

Psilocybin has been useful in treating mental states including depression and anxiety. With mood and anxiety disorders, three controlled trials have suggested that psilocybin may decrease symptoms of depression and anxiety in the context of cancer-related psychiatric distress for at least six months following a single acute administration. Psilocybin has also been shown to be useful in treating addiction. It has been reported that two to three moderate to high doses (20 and 30 mg/70 kg) of psilocybin, in combination with cognitive behavioral therapy (CBT) for smoking cessation, resulted in substantially higher six-month smoking abstinence rates than are typically observed with other medications or CBT alone.

The past two decades have witnessed growing interest in the clinical potential of psilocybin and other classic psychedelics to treat neuropsychiatric conditions including substance use disorders. ¹⁻⁶ Alcohol use disorder is a particularly promising target for treatment with psychedelics. A meta-analysis³⁹ of results from six randomized trials published between 1966 and 1971³³⁻³⁸ revealed that alcohol-dependent participants treated with lysergic acid diethylamide (LSD) were nearly twice as likely as those in comparator conditions to demonstrate remission during follow-up (odds ratio 1.96, 95% confidence interval 1.36-2.84, Z=3.59, p=0.0003).

There remains a need for more effective treatments for AUD.

### SUMMARY OF THE INVENTION

This invention provides methods for treating a human subject afflicted with alcohol use disorder (AUD) comprising administering to the subject an amount of a psilocybin-based compound sufficient to continuously treat the subject for at least 32 weeks.

### BRIEF DESCRIPTION OF THE FIGURES

FIG. 1 This figure presents a CONSORT flow diagram showing participant flow through each stage of the randomized controlled trial (enrollment, allocation, follow-up, and analysis).

FIG. 2 This figure presents the effects of treatment on continuous drinking outcomes. Mean and SE estimates for screening (84 days prior to screen), weeks 1-4 (28 days prior to first double-blind medication session; covariate in the model), and eight 28-day bins following the first double-blind medication session (shaded area: weeks 5-8, 9-12, 13-16, 17-20, 21-24, 25-28, 29-32, and 33-36). Arrows represent double-blind medication sessions 1 and 2; (a) main effect of treatment on percent heavy drinking days (primary outcome); (b) main effect of treatment on percent drinking days; and (c) main effect of treatment on drinks per day.

### DETAILED DESCRIPTION OF THE INVENTION

### Definitions

In this application, certain terms are used which shall have the meanings set forth as follows.

As used herein, “administer”, with respect to a psilocybin-based compound, means to deliver the compound to a subject's body via any known method suitable for that purpose. Specific modes of administration include, without limitation, oral administration and parenteral administration. Specifically envisioned in the present invention are, without limitation, oral administration, intravenous administration, subcutaneous administration, intra-arterial administration, intramuscular administration, intraperitoneal administration, intranasal administration, intrathecal administration, infusion, and administration via implant.

“Alcohol use disorder” is also referred to herein as “AUD”, and is also referred to in the art as “alcoholism”, “alcohol dependence”, and “alcohol addiction.”

As used herein, a “drink” shall mean 14 g of alcohol.

As used herein, a “drinking day” (also referred to as “DD”) shall mean a day during which a subject consumes any amount (even a sip) of alcohol.

As used herein, a “heavy drinking day” (also referred to as “HDD”) shall mean a day during which a subject consumes five or more drinks per day (if male) or four or more drinks per day (if female).

As used herein, a “human subject” can be of any age, gender, or state of co-morbidity. In one embodiment, the subject is male, and in another, the subject is female. In another embodiment, the subject is co-morbid (e.g., afflicted with both AUD and another disorder such as major depressive disorder (MDD) or a personality disorder (PD)). In a further embodiment, the subject is not co-morbid. In still another embodiment, the subject is younger than 20 years old, younger than 25 years old, younger than 30 years old, younger than 35 years old, younger than 40 years old, younger than 45 years old, younger than 50 years old, younger than 55 years old, or younger than 60 years old. In yet another embodiment, the subject is at least 60 years old, at least 65 years old, at least 70 years old, at least 75 years old, at least 80 years old, at least 85 years old, or at least 90 years old. In a further embodiment, the subject is refractory to treatment with one or more drugs. In one example, the subject is refractory to treatment with one or more of acamprosate, disulfiram, and naltrexone. In yet a further embodiment, the subject is refractory to psychotherapy. In still a further embodiment, the subject has been afflicted with

AUD for less than one year, between one and five years, between five and 10 years, between 10 and 15 years, between 15 and 20 years, or more than 20 years. In a further embodiment, the subject has an MEQ total score of <0.6 in response to 25 mg/70 kg of psilocybin. In yet a further embodiment, the subject has an MEQ total score of ≥0.6 in response to 25 mg/70 kg of psilocybin.

“Psilocybin” is also known in the art as (i) [3-[2-(dirnethylamino)ethyl]-1H-indo1-4-yl] dihydrogen phosphate and (ii) psilocybine.

As used herein, a “psilocybin-based compound” includes, without limitation, (i) psilocybin, (ii) a pharmaceutically acceptable salt of psilocybin, (iii) a deuterated form of psilocybin, (iv) a prodrug form of psilocybin, and (v) a metabolite of psilocybin (e.g., psilocin). Pharmaceutically acceptable salts of psilocybin include, without limitation, (i) anionic salts such as chloride (i.e., HCI), bromide, iodide, sulfate, nitrate, phosphate, citrate, methanesulfonate, trifluoroacetate, and acetate salts; (ii) cationic salts such as sodium, potassium, magnesium, calcium, and ammonium (e.g., tetramethylammonium) salts; (iii) acid salts such as acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate, and undecanoate salts; and (iv) basic salts such as alkali metal (e.g., sodium), alkaline earth metal (e.g., magnesium), ammonium, and N-(alkyl)₄ salts. Prodrug forms of psilocybin include, without limitation, esters. Deuterated forms of psilocybin, include, without limitation, psilocybin containing a single hydrogen-deuterium replacement, psilocybin containing two hydrogen-deuterium replacements, psilocybin containing three hydrogen-deuterium replacements, and psilocybin having all of its hydrogen atoms replaced by deuterium.

In this invention, the psilocybin-based compound (e.g., psilocybin) is preferably formulated using one or more routinely used pharmaceutically acceptable carriers. Such carriers are well known to those skilled in the art.

For example, oral drug delivery systems include tablets (e.g., a compressed preparation containing (i) 5-10% of the psilocybin-based compound, (ii) 80% of fillers, disintergrants, lubricants, glidants, and binders, and (iii) 10% of compounds which ensure easy disintegration, disaggregation, and dissolution of the tablet in the stomach or the intestine). Tablet dissolution time can be modified for a rapid effect or for sustained release. Special coatings can make the tablet resistant to stomach acid such that it only disintegrates in the duodenum, jejunum, and colon as a result of enzyme action or alkaline pH. Tablets can be coated with sugar, varnish, or wax to mask the drug's unpleasant taste. Oral drug delivery systems also include capsules (e.g., having a gelatinous envelope enclosing the psilocybin-based compound). Capsules can be designed to remain intact for some hours after ingestion to delay absorption, and may also contain both slow and fast release particles to produce rapid and sustained absorption in the same dose.

Injectable forms (e.g., solutions, suspensions, and emulsions) include, without limitation, sterile aqueous solutions or dispersions and sterile powders for reconstitution into sterile injectable solutions or dispersions. The carrier can be, for examples, a solvent or dispersing medium containing, for example, water, ethanol, a polyol (e.g., glycerol, propylene glycol, liquid polyethylene glycol, and the like), suitable mixtures thereof, and vegetable oils. Proper fluidity can be maintained, for example, by the use of a coating such as lecithin, by the maintenance of the required particle size in the case of dispersion and by the use of surfactants. Nonaqueous vehicles such as cottonseed oil, sesame oil, olive oil, soybean oil, corn oil, sunflower oil, or peanut oil and esters, such as isopropyl myristate, may also be used as solvent systems for compound compositions. Additionally, various additives that enhance the stability, sterility, and isotonicity of the compositions, including antimicrobial preservatives, antioxidants, chelating agents, and buffers, can be added. Prevention of microorganism action can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents for delaying absorption, for example, aluminum monostearate and gelatin.

Injectable forms can be administered parenterally in the form of slow-release subcutaneous implants or targeted delivery systems employing, for example, monoclonal antibodies, vectored delivery, polymer matrices, liposomes, and microspheres. Examples of delivery systems useful in the present invention include those disclosed in U.S. Pat. Nos. 5,225,182; 5,169,383; 5,167,616; 4,959,217; 4,925,678; 4,487,603; 4,486,194; 4,447,233; 4,447,224; and 4,439,196. Other such implants, delivery systems, and modules are well known to those skilled in the art.

As used herein, the term “subject” includes, without limitation, a mammal such as a human, a non-human primate, a dog, a cat, a horse, a sheep, a goat, a cow, a rabbit, a pig, a hamster, a rat, and a mouse. The present methods are envisioned for these non-human subjects, mutatis mutandis, as they are for human subjects in this invention.

As used herein, a “therapeutically effective amount” (e.g., an amount sufficient to continuously treat AUD in a subject for at least six weeks) of a psilocybin-based compound (e.g., psilocybin) includes, without limitation, (i) 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, or 70 mg; (ii) 1 mg to 5 mg, 5 mg to 10 mg, 10 mg to 15 mg, 15 mg to 20 mg, 20 mg to 25 mg, 25 mg to 30 mg, 30 mg to 35 mg, 35 mg to 40 mg, 40 mg to 45 mg, 45 mg to 50 mg, 50 mg to 55 mg, 55 mg to 60 mg, 60 mg to 65 mg, or 65 mg to 70 mg; (iii) 10 μg/kg, 20 μg/kg, 30 μg/kg, 40 μg/kg, 50 μg/kg, 60 μg/kg, 70 μg/kg, 80 μg/kg, 90 μg/kg, 100 μg/kg, 110 μg/kg, 120 μg/kg, 130 μg/kg, 140 μg/kg, 150 μg/kg, 160 μg/kg, 170 μg/kg, 180 μg/kg, 190 μg/kg, 200 μg/kg, 210 μg/kg, 220 μg/kg, 230 μg/kg, 240 μg/kg, 250 μg/kg, 260 μg/kg, 270 μg/kg, 280 μg/kg, 290 μg/kg, 300 μg/kg, 310 μg/kg, 320 μg/kg, 330 μg/kg, 340 μg/kg, 350 μg/kg, 360 μg/kg, 370 μg/kg, 380 μg/kg, 390 μg/kg, 400 μg/kg, 410 μg/kg, 420 μg/kg, 430 μg/kg, 440 μg/kg, 450 μg/kg, 460 μg/kg, 470 μg/kg, 480 μg/kg, 490 μg/kg, 500 μg/kg, 510 μg/kg, 520 μg/kg, 530 μg/kg, 540 μg/kg, 550 μg/kg, 560 μg/kg, 570 μg/kg, 580 μg/kg, 590 μg/kg, 600 μg/kg, 610 μg/kg, 620 μg/kg, 630 μg/kg, 640 μg/kg, 650 μg/kg, 660 μg/kg, 670 μg/kg, 680 μg/kg, 690 μg/kg, or 700 μg/kg; (iv) μg/kg to 50 μg/kg, 50 μg/kg to 100 μg/kg, 100 μg/kg to 150 μg/kg, 150 μg/kg to 200 μg/kg, 200 μg/kg to 250 μg/kg, 250 μg/kg to 300 μg/kg, 300 μg/kg to 350 μg/kg, 350 μg/kg to 400 μg/kg, 400 μg/kg to 450 μg/kg, 450 μg/kg to 500 μg/kg, 500 μg/kg to 550 μg/kg, 550 μg/kg to 600 μg/kg, 600 μg/kg to 650 μg/kg, or 650 μg/kg to 700 μg/kg; (v) 10 mg/70 kg, 11 mg/70 kg, 12 mg/70 kg, 13 mg/70 kg, 14 mg/70 kg, 15 mg/70 kg, 16 mg/70 kg, 17 mg/70 kg, 18 mg/70 kg, 19 mg/70 kg, 20 mg/70 kg, 21 mg/70 kg, 22 mg/70 kg, 23 mg/70 kg, 24 mg/70 kg, 25 mg/70 kg, 26 mg/70 kg, 27 mg/70 kg, 28 mg/70 kg, 29 mg/70 kg, 30 mg/70 kg, 31 mg/70 kg, 32 mg/70 kg, 33 mg/70 kg, 34 mg/70 kg, 35 mg/70 kg, 36 mg/70 kg, 37 mg/70 kg, 38 mg/70 kg, 39 mg/70 kg, 40 mg/70 kg, 41 mg/70 kg, 42 mg/70 kg, 43 mg/70 kg, 44 mg/70 kg, or 45 mg/70 kg; or (vi) 10 mg/70 kg to 15 mg/70 kg, 15 mg/70 kg to 20 mg/70 kg, 20 mg/70 kg to 25 mg/70 kg, 25 mg/70 kg to 30 mg/70 kg, 30 mg/70 kg to 35 mg/70 kg, 35 mg/70 kg to 40 mg/70 kg, or 40 mg/70 kg to 45 mg/70 kg.

As used herein, “treating” a subject afflicted with AUD includes, without limitation, doing one or more of the following: (i) causing a continuous reduction in the percent of heavy drinking days (e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) fora period of time (e.g., at least 32 weeks, at least 40 weeks, at least one year, at least two years, or at least three years); (ii) causing a continuous reduction in the percent of drinking days (e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) for a period of time (e.g., at least 32 weeks, at least 40 weeks, at least one year, at least two years, or at least three years); (iii) causing a continuous reduction in drinks per day (e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) for a period of time (e.g., at least 32 weeks, at least 40 weeks, at least one year, at least two years, or at least three years); and (iv) causing a continuous reduction in World Health Organization risk level⁴⁸ by one, two, or three levels (preferably at least two levels) for a period of time (e.g., at least 32 weeks, at least 40 weeks, at least one year, at least two years, or at least three years).

By way of example, a “continuous” reduction in the percent of heavy drinking days for a period of time occurs when the percent of heavy drinking days for a subject after psilocybin-based compound administration is lower during each portion of the period of time than for a subject who did not receive that compound. Preferably, the degree of this reduction does not diminish during the period of time. By way of additional example, a continuous reduction in the percent of drinking days for a period of time occurs when the percent of drinking days for a subject after psilocybin-based compound administration is lower during each portion of the period of time than for a subject who did not receive that compound. Preferably, the degree of this reduction does not diminish during the period of time. By way of further example, a continuous reduction in drinks per day for a period of time occurs when drinks per day for a subject after psilocybin-based compound administration is lower on each day during the period of time than for a subject who did not receive that compound.

Preferably, the degree of this reduction does not diminish during the period of time.

Preferably, treating a subject afflicted with AUD includes (i) causing a continuous reduction in the percent of heavy drinking days for a period of time (e.g., at least 32 weeks, at least 40 weeks, at least one year, at least two years, or at least three years); (ii) causing a continuous reduction in the percent of drinking days for a period of time (e.g., at least 32 weeks, at least 40 weeks, at least one year, at least two years, or at least three years); or (iii) causing a continuous reduction in drinks per day for a period of time (e.g., at least 32 weeks, at least 40 weeks, at least one year, at least two years, or at least three years). More preferably, treating a subject afflicted with AUD includes (i) causing a continuous reduction in the percent of heavy drinking days for a period of time (e.g., at least 32 weeks, at least 40 weeks, at least one year, at least two years, or at least three years); (ii) causing a continuous reduction in the percent of drinking days for a period of time (e.g., at least 32 weeks, at least 40 weeks, at least one year, at least two years, or at least three years); and (iii) causing a continuous reduction in drinks per day for a period of time (e.g., at least 32 weeks, at least 40 weeks, at least one year, at least two years, or at least three years). More preferably, treating a subject afflicted with AUD includes doing one or more of the following: (i) causing the continuous elimination of heavy drinking days for a period of time (e.g., at least 32 weeks, at least 40 weeks, at least one year, at least two years, or at least three years); (ii) causing the continuous elimination of drinking days for a period of time (e.g., at least 32 weeks, at least 40 weeks, at least one year, at least two years, or at least three years); and (iii) causing the continuous elimination of drinks per day for a period of time (e.g., at least 32 weeks, at least 40 weeks, at least one year, at least two years, or at least three years). Even more preferably, treating a subject afflicted with AUD includes (i) causing the continuous elimination of heavy drinking days for a period of time (e.g., at least 32 weeks, at least 40 weeks, at least one year, at least two years, or at least three years); (ii) causing the continuous elimination of drinking days for a period of time (e.g., at least 32 weeks, at least 40 weeks, at least one year, at least two years, or at least three years); and (iii) causing the continuous elimination of drinks per day for a period of time (e.g., at least 32 weeks, at least 40 weeks, at least one year, at least two years, or at least three years). Ideally, treating a subject afflicted with AUD causes continuous abstinence for a period of time (e.g., at least 32 weeks, at least 40 weeks, at least one year, at least two years, or at least three years), and preferably permanently.

In a further preferred embodiment, treating a subject afflicted with AUD comprises causing a continuous reduction in the percent of heavy drinking days for at least 32 weeks, whereby the subject's percent of heavy drinking days after the first 32 weeks following psilocybin administration is below 50% (or below 45%, 40%, 35%, 30%, 25%, 20%, 19%, 18%, 17%, 16%, or 15%) of what the subject's percent of heavy drinking days was five weeks prior to psilocybin administration. For instance, in on embodiment, treating a subject afflicted with AUD comprises causing a continuous reduction in the percent of heavy drinking days for at least 32 weeks, whereby the subject's percent of heavy drinking days after the first 32 weeks following psilocybin administration is below 25% of what the subject's percent of heavy drinking days was five weeks prior to psilocybin administration. In a further preferred embodiment, treating a subject afflicted with AUD comprises causing a continuous reduction in the percent of drinking days for at least 32 weeks, whereby the subject's percent of drinking days after the first 32 weeks following psilocybin administration is below 60% (or below 55%, 50%, 45%, 44%, 43%, 42%, 41%, 40%, 39%, 38%, 37%, 36%, or 35%) of what the subject's percent of drinking days was five weeks prior to psilocybin administration. For instance, in on embodiment, treating a subject afflicted with AUD comprises causing a continuous reduction in the percent of drinking days for at least 32 weeks, whereby the subject's percent of drinking days after the first 32 weeks following psilocybin administration is below 45% of what the subject's percent of drinking days was five weeks prior to psilocybin administration.

In yet a further preferred embodiment, treating a subject afflicted with AUD comprises causing a continuous reduction in drinks per day for at least 32 weeks, whereby the subject's drinks per day after the first 32 weeks following psilocybin administration is below 45% (or below 40%, 35%, 30%, 25%, 24%, 23%, 22%, 21%, or 20%) of what the subject's drinks per day was five weeks prior to psilocybin administration. For instance, in on embodiment, treating a subject afflicted with AUD comprises causing a continuous reduction in drinks per day for at least 32 weeks, whereby the subject's drinks per day after the first 32 weeks following psilocybin administration is below 30% of what the subject's drinks per day was five weeks prior to psilocybin administration.

In another preferred embodiment, treating a subject afflicted with AUD comprises causing a continuous reduction in the percent of heavy drinking days for at least 32 weeks, whereby the subject's percent of heavy drinking days after the first 32 weeks following psilocybin administration is below 60% (or below 55%, 50%, 45%, 44%, 43%, 42%, 41%, 40%, 39%, 38%, 37%, 36%, or 35%) of what the subject's percent of heavy drinking days was one day prior to psilocybin administration. For instance, in on embodiment, treating a subject afflicted with AUD comprises causing a continuous reduction in the percent of heavy drinking days for at least 32 weeks, whereby the subject's percent of heavy drinking days after the first 32 weeks following psilocybin administration is below 45% of what the subject's percent of heavy drinking days was one day prior to psilocybin administration. In a further preferred embodiment, treating a subject afflicted with AUD comprises causing a continuous reduction in the percent of drinking days for at least 32 weeks, whereby the subject's percent of drinking days after the first 32 weeks following psilocybin administration is below 70% (or below 65%, 60%, 59%, 58%, 57%, 56%, 55%, 54%, 53%, 52%, 51%, or 50%) of what the subject's percent of drinking days was one day prior to psilocybin administration. For instance, in on embodiment, treating a subject afflicted with AUD comprises causing a continuous reduction in the percent of drinking days for at least 32 weeks, whereby the subject's percent of drinking days after the first 32 weeks following psilocybin administration is below 60% of what the subject's percent of drinking days was one day prior to psilocybin administration. In yet a further preferred embodiment, treating a subject afflicted with AUD comprises causing a continuous reduction in drinks per day for at least 32 weeks, whereby the subject's drinks per day after the first 32 weeks following psilocybin administration is below 55% (or below 50%, 45%, 44%, 43%, 42%, 41%, 40%, 39%, 38%, 37%, 36%, or 35%) of what the subject's drinks per day was one day prior to psilocybin administration. For instance, in on embodiment, treating a subject afflicted with AUD comprises causing a continuous reduction in drinks per day for at least 32 weeks, whereby the subject's drinks per day after the first 32 weeks following psilocybin administration is below 45% of what the subject's drinks per day was one day prior to psilocybin administration.

### Embodiments of the Invention

This invention provides a method for treating a subject (preferably human) afflicted with alcohol use disorder (AUD) comprising administering to the subject a therapeutically effective amount of a psilocybin-based compound (e.g., an amount of a psilocybin-based compound sufficient to continuously treat the subject for at least six weeks).

In a preferred embodiment of the present method, the psilocybin-based compound is psilocybin. In another preferred embodiment of the present method, the administering is oral (e.g., via capsule).

In one embodiment of the present method, the method comprises administering to the subject a single one-time dose (i.e., a therapeutically effective amount) of the psilocybin-based compound (e.g., psilocybin). For example, in one preferred embodiment of the present method, the method comprises administering to the subject (preferably abstinent for at least one day prior to administration) a single one-time dose of the psilocybin-based compound, wherein the dose is from 25 mg/70 kg to 40 mg/70 kg. In another preferred embodiment of the present method, the method comprises administering to the subject a single one-time dose of the psilocybin-based compound, wherein the dose is less than 25 mg/70 kg. This dose can be self-administered (e.g., orally via a capsule) alone or, preferably, in the presence of a healthcare provider (e.g., during an observation period of four to eight hours). Administering the dose in the presence of a healthcare provider is advantageous in that it permits the healthcare provider to monitor the subject for adverse effects and, if necessary, administer additional aid (e.g., diazepam for acute anxiety). In that regard, and in a preferred embodiment, the present method comprises (i) administering psilocybin to the subject as set forth herein and (ii) concurrently (e.g., beginning on the day of psilocybin administration and, ideally, 30-60 minutes prior to psilocybin administration) administering to the subject an anti-anxiety drug (e.g., diazepam (sold as Valium®)). For example, in one embodiment, the administration of each dose of psilocybin is accompanied by the oral administration of diazepam at (i) 2 mg to 10 mg once (e.g., 2 mg, 3 mg, 4 mg, 5 mg, 6 mg, 7 mg, 8 mg, 9 mg, or 10 mg once), or 2-4 times daily, or (ii) 10 mg once, or 3 or 4 times during first 24 hours, then 5 mg, 3 or 4 times daily as needed.

In another embodiment of the present method, the method comprises administering to the subject two doses (i.e., two therapeutically effective amounts) of the psilocybin-based compound (e.g., psilocybin) separated by a suitable period of time (e.g., one week, two weeks, three weeks, four weeks, five weeks, six weeks, seven weeks, or eight weeks). These doses can be self-administered (e.g., orally via a capsule) alone or, preferably, in the presence of a healthcare provider (e.g., during an observation period of four to eight hours). In one embodiment, the first and second doses are the same. In another embodiment, the second dose is greater than the first dose (e.g., wherein the second dose is 10%, 20%, 30%, 40%, 50%, 60%, 70%, or 80% greater than the first dose). For example, in one preferred embodiment of the present method, the method comprises administering to the subject (preferably abstinent for at least one day prior to each administration) two doses of the psilocybin-based compound, wherein (i) the doses are administered four weeks apart, and (ii) each dose is from 25 mg/70 kg to 40 mg/70 kg.

In another preferred embodiment of the present method, the subject experiences a continuous reduction in the percent of heavy drinking days for a period of at least 32 weeks (e.g., at least 40 weeks, at least one year, at least two years, or at least three years) following administration of the first (or only) dose. In yet another preferred embodiment of the present method, the subject experiences a continuous reduction in the percent of drinking days for a period of at least 32 weeks (e.g., at least 40 weeks, at least one year, at least two years, or at least three years) following administration of the first (or only) dose. In a further preferred embodiment of the present method, the subject experiences a continuous reduction in drinks per day for a period of at least 32 weeks (e.g., at least 40 weeks, at least one year, at least two years, or at least three years) following administration of the first (or only) dose.

In a preferred embodiment of the present method, the subject undergoes psychotherapy prior to, concurrently with, and/or following psilocybin administration. This embodiment is described in detail below.

The following additional embodiments of the present method are exemplary.

In a first embodiment, this invention provides a method for treating a human subject afflicted with AUD comprising orally administering to the subject two doses of psilocybin (e.g., in capsule form), wherein (i) the doses are administered four weeks apart, and (ii) each dose is 300 μg/kg to 350 μg/kg.

In a second embodiment, this invention provides a method for treating a human subject afflicted with AUD comprising orally administering to the subject two doses of psilocybin (e.g., in capsule form), wherein (i) the doses are administered four weeks apart, and (ii) each dose is 250 μg/kg to 300 μg/kg.

In a third embodiment, this invention provides a method for treating a human subject afflicted with AUD comprising orally administering to the subject two doses of psilocybin (e.g., in capsule form), wherein (i) the doses are administered four weeks apart, and (ii) each dose is 200 μg/kg to 250 μg/kg.

In a fourth embodiment, this invention provides a method for treating a human subject afflicted with AUD comprising orally administering to the subject two doses of psilocybin (e.g., in capsule form), wherein (i) the doses are administered four weeks apart, and (ii) each dose is 150 μg/kg to 200 μg/kg.

In a fifth embodiment, this invention provides a method for treating a human subject afflicted with AUD comprising orally administering to the subject two doses of psilocybin (e.g., in capsule form), wherein (i) the doses are administered four weeks apart, and (ii) each dose is 100 μg/kg to 150 μg/kg.

In a sixth embodiment, this invention provides a method for treating a human subject afflicted with AUD comprising orally administering to the subject two doses of psilocybin (e.g., in capsule form), wherein (i) the doses are administered four weeks apart, and (ii) each dose is 50 μg/kg to 100 μg/kg.

In a seventh embodiment, this invention provides a method for treating a human subject afflicted with AUD comprising orally administering to the subject a single once-only dose of psilocybin (e.g., in capsule form), wherein the dose is 300 μg/kg to 350 μg/kg.

In an eighth embodiment, this invention provides a method for treating a human subject afflicted with AUD comprising orally administering to the subject a single once-only dose of psilocybin (e.g., in capsule form), wherein the dose is 250 μg/kg to 300 μg/kg.

In a ninth embodiment, this invention provides a method for treating a human subject afflicted with AUD comprising orally administering to the subject a single once-only dose of psilocybin (e.g., in capsule form), wherein the dose is 200 μg/kg to 250 μg/kg.

In a tenth embodiment, this invention provides a method for treating a human subject afflicted with AUD comprising orally administering to the subject a single once-only dose of psilocybin (e.g., in capsule form), wherein the dose is 150 μg/kg to 200 μg/kg.

In an eleventh embodiment, this invention provides a method for treating a human subject afflicted with AUD comprising orally administering to the subject a single once-only dose of psilocybin (e.g., in capsule form), wherein the dose is 100 μg/kg to 150 μg/kg.

In a twelfth embodiment, this invention provides a method for treating a human subject afflicted with AUD comprising orally administering to the subject a single once-only dose of psilocybin (e.g., in capsule form), wherein the dose is 50 μg/kg to 100 μg/kg.

The present methods are envisioned for psilocybin-based compounds other than psilocybin, mutatis mutandis, as they are for psilocybin in this invention.

This invention also provides a composition suitable for treating AUD comprising a therapeutically effective amount of a psilocybin-based compound (e.g., psilocybin) and a pharmaceutically effective carrier. Preferably, the present composition is formulated at one of the doses exemplified herein using one of the pharmaceutically acceptable carriers exemplified herein.

This invention will be better understood by reference to the examples which follow, but those skilled in the art will readily appreciate that the specific examples detailed are only illustrative of the invention as described more fully in the claims which follow thereafter.

### EXAMPLE 1

The following describes a multi-site randomized controlled trial conducted to evaluate the efficacy of psilocybin-assisted treatment of alcohol use disorder. Reported here are drinking outcomes for the double-blind phase of this trial.

### I—Key Points

Question: Does psilocybin-assisted treatment improve drinking outcomes in alcohol use disorder patients, relative to outcomes observed with active placebo medication?

Findings: In this double-blind, randomized controlled trial with 93 treated participants, the percentage of heavy drinking days during 32 weeks of follow-up was 9.7 for the psilocybin group and 23.6 for the diphenhydramine group, a significant difference. Average daily alcohol consumption (number of drinks per day) was also significantly lower in the psilocybin group.

Meaning: Psilocybin administered in combination with psychotherapy produced robust decreases in drinking over and above those produced by active placebo and psychotherapy in this trial.

### II—Abstract

Importance: Although classic psychedelic medications have shown promise in the treatment of alcohol use disorder (AUD), the efficacy of psilocybin remains unknown.

Objective: To determine whether two administrations of high-dose psilocybin improve drinking outcomes in AUD patients relative to outcomes observed with active placebo medication.

Design: In this double-blind trial, participants were offered 12 weeks of manualized psychotherapy and were randomly assigned to receive psilocybin vs. diphenhydramine during two day-long medication sessions at week 4 and 8. Outcomes were assessed over the 32-week double-blind period following the first dose of study medication.

Setting: The study was conducted at two academic centers. Participants were recruited from the community over an approximately six-year period.

Participants: Adults between age 25 and 65 with a DSM-IV diagnosis of alcohol dependence and at least 4 heavy drinking days during the 30 days prior to screening.

Interventions: Study medications were psilocybin 25 mg/70 kg vs. diphenhydramine 50 mg (first session) and psilocybin 25-40 mg/70 kg vs. diphenhydramine 50-100 mg (second session). Psychotherapy included motivational enhancement therapy and cognitive-behavioral therapy.

Main Outcomes and Measures: The a priori primary outcome was percent heavy drinking days, assessed using the timeline followback interview, contrasted between groups over the 32-week period following the first administration of study medication, using multivariate repeated measures analysis of variance.

Results: 95 participants (mean age=46 [SD=12] years; 44% female) were randomized. 93 participants received at least one dose of study medication and were included in the primary outcome analysis. Percent heavy drinking days during the 32-week double-blind period was 9.7 for the psilocybin group and 23.6 for the diphenhydramine group, a mean difference of 13.9 (95% Cl: 3.0-24.7; F_{(1, 86)}=6.43, p=0.013). Average daily alcohol consumption (number of standard drinks per day) was also lower in the psilocybin group. There were no serious adverse events among participants who received psilocybin.

Conclusions and Relevance: Psilocybin administered in combination with psychotherapy produced robust decreases in drinking over and above those produced by active placebo and psychotherapy. These results provide strong support for further study of psilocybin-assisted treatment for AUD.

Trial Registration: clinicaltrials.gov NCT#02061293

### III—Introduction

The past two decades have witnessed growing interest in the clinical potential of psilocybin and other classic psychedelics to treat neuropsychiatric conditions including substance use disorders.¹⁻⁸ Although the mechanisms of psychedelic-assisted treatments remain unclear, the action of these drugs at the serotonin 2A receptor and down-stream effects on neurotransmission, intracellular signaling, epigenetics, and gene expression appear to enhance plasticity at multiple levels, including neuronal structure, neural networks, cognition, affect, and behavior.⁹⁻²⁴ Some clinically relevant effects may be independent of serotonin 2A receptor activation.²⁴⁻²⁵ The direction and magnitude of change observed in a therapeutic context is influenced by the subjective experience under the influence of the drug,²⁶⁻²⁹ and by contextual factors, including concomitant psychotherapy.³⁰⁻³

Alcohol use disorder (AUD) is a particularly promising target for treatment with psychedelics. A meta-analysis of results from six randomized trials published between 1966 and 1971³³⁻³⁸ revealed that alcohol-dependent participants treated with lysergic acid diethylamide (LSD) were nearly twice as likely as those in comparator conditions to demonstrate remission during follow-up (odds ratio 1.96, 95% confidence interval 1.36-2.84, Z=3.59, p=0.0003).³⁹ An open-label study published in 2015 demonstrated that moderately high doses of psilocybin (21-28 mg/70 kg) were well tolerated by alcohol-dependent participants, and large reductions in drinking were observed over a 32-week follow-up period.³

This multi-site randomized controlled trial evaluated the efficacy of psilocybin-assisted treatment of AUD. Here we report drinking outcomes for the double-blind phase of the trial.

### IV—Methods

### Trial Oversight

The study was reviewed and approved by the Heffter Research Institute, the institutional review boards of each site (NYU Grossman School of Medicine and the University of New Mexico Health Sciences Center), the United States Food and Drug Administration and Drug Enforcement Administration, the New Mexico Board of Pharmacy, and the New York State Bureau of Narcotics Enforcement. Psilocybin was provided by the Usona Institute, Dr. Nicholas Cozzi at the University of Wisconsin-Madison, and Dr. David Nichols at Purdue University. The study was overseen by a data and safety monitoring board. The first author was the IND holder for the trial. This report follows the Consolidated Standards of Reporting Trials (CONSORT) guideline for parallel-group randomized trials.

### Participants

Participants were recruited over an approximately six-year period using advertisements in local media, and provided written informed consent. Participants were between age 25 and 65, had a diagnosis of alcohol dependence ascertained using the Structured Clinical Interview for DSM-IV⁴⁰ and had at least 4 heavy drinking days during the 30 days prior to screening (heavy drinking days were defined as 5 or more drinks in a day for a man, 4 or more drinks in a day for a woman). Exclusion criteria included major psychiatric and drug use disorders; any hallucinogen use in the past year, or >25 lifetime uses; medical conditions that contraindicated either of the study medications; use of exclusionary medications; and current treatment for AUD.

### Trial Design

### Overview

Qualifying participants were assessed at screening, baseline (week 0), and weeks 4, 5, 8, 9, 12, 24, and 36. They were randomly assigned in a 1:1 ratio to receive either psilocybin or diphenhydramine, administered in two eight-hour sessions at weeks 4 and 8. All participants who completed the double-blind observation period (weeks 5-36) and still met safety criteria were offered an open-label psilocybin session at week 38, including four additional psychotherapy sessions and assessment for an additional 18 weeks. Participants received up to a total of $560 for completing assessments in the course of the trial, but were not reimbursed for attending the therapy and medication sessions.

### Psychotherapeutic Elements of Treatment

All participants were offered a total of 12 psychotherapy sessions from a team of two therapists including a licensed psychiatrist: 4 before the first medication session, 4 between the first and second medication sessions, and 4 in the month following the second medication session. The psychotherapy, described in detail in a separate publication,⁴¹ included motivational interviewing and cognitive behavioral therapy for AUD as well as material designed to help the participants to manage and make use of the psychoactive effects of the study medication.

### Randomization and Blinding

Randomization was stratified by site and consisted of balanced blocks of varying size. A study pharmacist at each site generated the randomization sequence and assigned treatment in order of randomization. All other study staff and investigators as well as participants were blinded to treatment assignment.

### Dosage of Study Medication

Study medication was taken orally in a single opaque capsule of unvarying appearance and weight. Psilocybin doses were weight-based to control for participant body weight, which ranged from 49.0 to 116.1 (mean=78.3, SD=15.6) kg. Doses for the first session were psilocybin 25 mg/70 kg or diphenhydramine 50 mg. Participants received an increased dose in the second session if there were no dose-limiting adverse events and they agreed to the increase. The increased dose of psilocybin was 30 mg/70 kg if the participant's total score on the Pahnke-Richards Mystical Experience Questionnaire (MEQ)⁴² was ≥0.6 in the first session (indicating a robust subjective response to the 25 mg/70 kg dose), or 40 mg/70 kg if the MEQ total score in the first session was <0.6. The increased dose of diphenhydramine was 100 mg, regardless of subjective response.

### Administration of Study Medication

Study medication was administered at approximately 9 AM, after which participants were required to stay in the session room with the therapists for at least 8 hours (except for bathroom breaks). During the session, participants were encouraged to lie on a couch wearing eyeshades and headphones providing a standardized playlist of music. Medications were available in the session room to treat hypertension, severe anxiety, or psychotic symptoms.

### Outcomes and Assessments

Measurements from this experiment are described below.

### Subjective Effects of Study Medication

Subjective effects of psilocybin vs. diphenhydramine were assessed using the States of Consciousness Questionnaire,⁴² containing the 43-item Mystical Experience Questionnaire (MEQ), completed immediately after each medication session.

### Drinking Outcomes

The pre-specified primary drinking outcome was the percentage of heavy drinking days (PH DD) during weeks 5-32, assessed at weeks 8, 12, 24, and 36 using the timeline followback, a reliable and valid calendar-based method which is the gold standard outcome for AUD clinical trials.⁴³⁻⁴⁷ Secondary outcomes included percent drinking days (PDD), average drinks per day (DPD), and dichotomous outcomes: abstinence, lack of heavy drinking days, and reduction in World Health Organization risk level⁴⁸ by one, two, or three levels. Hair or fingernail samples were collected at week 24 and assayed for ethylglucuronide (EtG) concentration to confirm self-reported abstinence. The Short Index of Problems (SIP-2R)⁴⁹ was used to assess drinking-related problems at baseline and weeks 12, 24, and 36.

### Safety

Blood pressure and heart rate were assessed at 30- to 60-minute intervals during the first 6 hours of each medication session. Adverse events were solicited at each post-screening assessment.

### Blinding Integrity

After each session, participants and therapists were asked to guess which medication had been administered, and rate their degree of certainty on a 100-point visual analog scale (0=not at all confident, 100=extremely confident).

### Statistical Analysis

The Statistical Analysis Plan was developed in accordance with published guidelines 50 and contains a full description of statistical methods.

### Sample Size and Power

The study was originally designed to randomize up to 180 participants. An interim analysis was planned after recruitment of 100 participants to re-estimate the necessary sample size to yield power of 0.8 to detect a small to moderate effect (f²=0.16), with no correction for multiple comparisons. However, following an indefinite mandatory suspension of recruitment, enrollment for this trial was halted at 95 randomized participants.

### Subjective Effects

MEQ scores for the first and second medication sessions were computed and contrasted by group (psilocybin vs. diphenhydramine) using t-tests for independent samples.

### Efficacy

To evaluate the effects of treatment on continuous drinking outcomes (PHDD, PDD, and DPD), 3-dimensional multivariate repeated-measures analysis of variance (RM 25 MANOVA) was used, including fixed, categorical effects of treatment, assessment, and site; site-by-treatment and treatment-by-assessment interactions; fixed baseline covariates for each dependent measure (PHDD, PDD, and DPD during weeks 1-4); and monthly values of PHDD, PDD, and DPD (weeks 5-8, 9-12, 13-16, 17-20, 21-24, 25-28, 29-32, 32-36) as a nested multivariate dependent measure. All missing monthly values of PHDD, PDD, and DPD were imputed simultaneously using Multivariate Imputation by Chained Equations in R (MICE v3.14.0).⁵¹ Significant multivariate treatment effects were decomposed with univariate repeated-measures F-tests within each drinking dimension (PHDD, PDD and DPD).⁵²

Treatment contrasts for dichotomous outcomes were obtained using Chi-squared statistics. Effects of treatment on problems related to drinking were compared using univariate mixed models for repeated measures (MMRM) and generalized linear models. Hedges'g was computed as a measure of effect size for between- and within-group differences on continuous outcomes, and odds ratios were computed for dichotomous outcomes. No correction was made for multiple comparisons, so analyses of secondary outcomes should be considered exploratory.

### Safety Outcomes

Blood pressure and heart rate treatment contrasts were based on MMRM with fixed, categorical effects of treatment and assessment, a treatment-by-assessment interaction, and a fixed covariate (value of each outcome prior to drug administration). All adverse events occurring after informed consent were MedDRA-coded and tabulated, and prevalence within treatment groups (proportion of participants affected) was compared using Fisher's exact tests.

### V—Results

### Participants

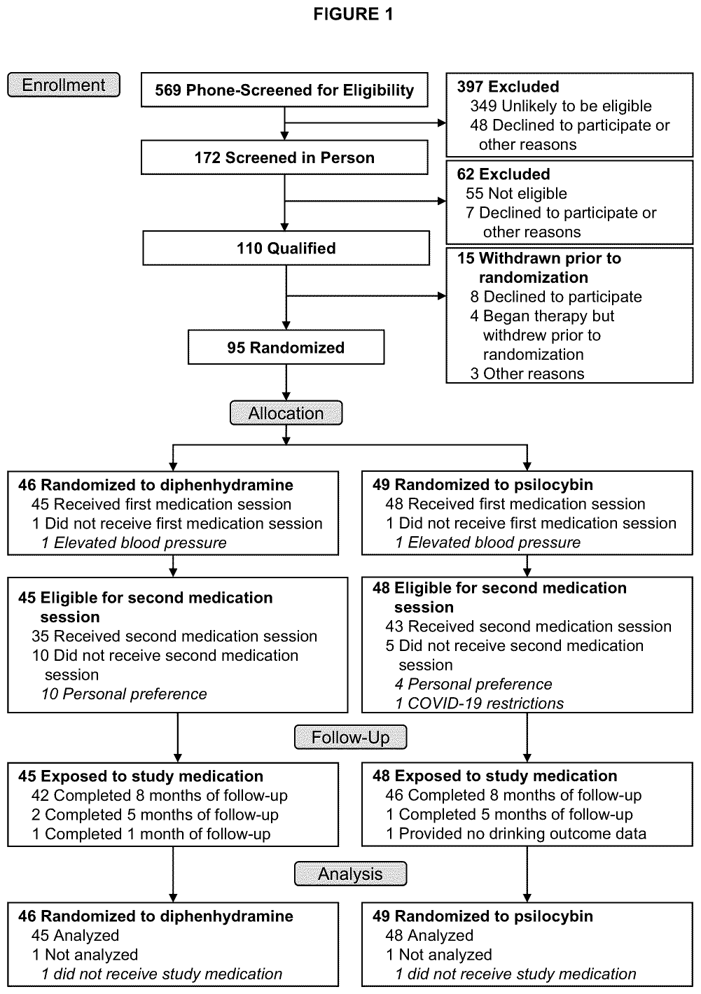

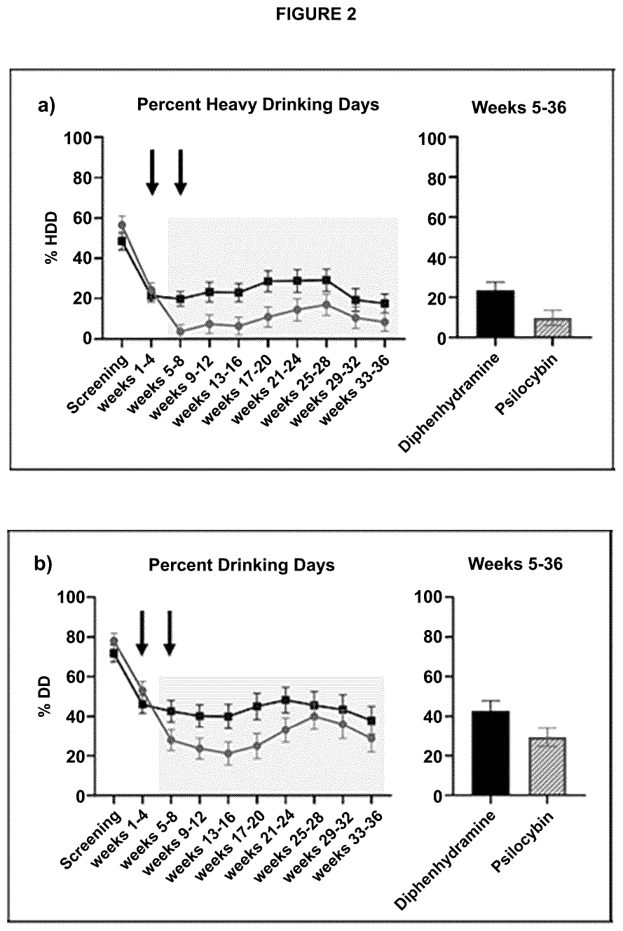

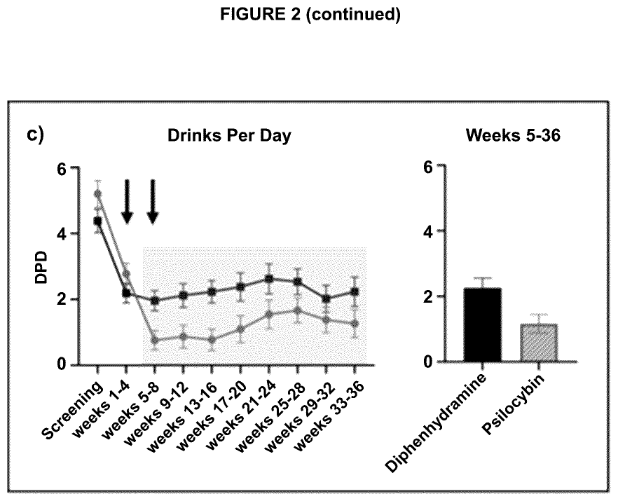

FIG. 1 summarizes recruitment of participants, treatment exposure, and retention. Ninety-five participants were randomized: 49 to psilocybin and 46 to diphenhydramine. Table 1 describes baseline characteristics of the randomized sample. Participants averaged age 45.8 (SD=11.6), 56% were male, and 78% were non-Hispanic Whites. They met 5.3 (SD=1.2) of the 7 alcohol dependence criteria and had been alcohol dependent for 14.2 (SD=9.7) years. During the 12 weeks prior to screening, they were drinking on 74.9% (SD=28.1%) of days, drinking heavily on 52.7% of days (SD=30.58), and averaging 7.1 (SD=4.1) standard drinks per drinking day.

### Treatment Exposure and Retention

Participation in the non-medication therapy sessions was high and did not substantially differ between the treatment groups. Psilocybin- and diphenhydramine-treated participants completed a mean of 11.75 (SD=0.76) and 11.47 (SD=1.20) of the 12 sessions, respectively (F_{(1,91)}=1.88, p=0.17). Ninety-three participants received at least 1 dose of medication: 48 received psilocybin and 45 received diphenhydramine. Forty-three of the psilocybin-treated participants (89.6%) and 35 of the 45 diphenhydramine-treated participants (77.8%) received a second double-blind medication session (F_{(1,91)}=2.40, p=0.13). In the second session, psilocybin doses were 25 mg/70 kg (n=1), 30 mg/70 kg (n=27), and 40 mg/70 kg (n =15); diphenhydramine doses were 50 mg (n=11), and 100 mg (n=24). Mean absolute dosages of psilocybin were 28.3 (SD=5.4, range 19.3-40.0) mg for psilocybin session 1, and 37.7 (SD=8.6, range 24.1-64.5) mg for psilocybin session 2.

Valid drinking outcome data were obtained for 717 out of 744 months (96.4%) in the 8-month follow-up period for the 93 treated participants (366/384 (95.3%) for the psilocybin group, 351/360 (97.5%) for the diphenhydramine group). 18.7% percent of follow-up TLFB assessments were collected by phone due to inability to complete in-person visits. EtG results were available for 50/93 participants (53.8%), with missing data due to telephone visits (n=24), insufficient hair samples (n=12), missing visits (n=5), or other reasons (n=2). Participants missing EtG data did not differ from other participants on baseline drinking measures, age, ethnicity, or sex (p>0.1).

### Blinding Integrity

Participants correctly guessed their treatment assignment in 93.6% of the first sessions, reporting a certainty of 88.5% (SD=23.2%). In the second session, 94.7% guessed correctly, and certainty was 90.6% (SD=21.5%). Study therapists correctly guessed treatment 92.4% of the time for first sessions and 97.4% for second sessions, and their certainties were 92.8% (SD=16.3%) and 95.4% (SD=2.9%), respectively.

### Convergent Validity of Self-Report and EtG

Among the 50 participants for whom valid EtG results were obtained at week 24, 14 (28%) reported total abstinence on the week 24 TLFB. EtG results were negative (<8 pg/ng) for all of these participants, providing some objective support for the veracity of self-report in this sample.

### Acute effects

### Cardiovascular Effects

Psilocybin administration was associated with increased systolic and diastolic blood pressure relative to diphenhydramine, but no participant reported symptoms or was treated for hypertension. By 360 minutes, blood pressure was no longer significantly elevated. Heart rate was also higher in the psilocybin group until approximately 300 minutes after drug administration.

### Subjective Effects

Mean MEQ scores for Session 1 were 0.59 (SD=0.24) in psilocybin-treated participants vs. 0.10 (SD=0.13) in those receiving diphenhydramine (t_{(1, 74.3)}=12.41, p<0.001). For Session 2, mean scores were 0.64 (SD=0.21), vs. 0.11 (SD=0.16), respectively (t_{(1, 75.5)}=13.01, p<0.001). These scores indicate high average intensity of experiences in the psilocybin group, and low average intensity in the diphenhydramine group.

### Changes in Drinking Prior to Randomization

Substantial decreases in PHDD, PDD and DPD were observed in both treatment groups between screening and week 4, during which time participants received 4 psychotherapy sessions and attempted to stop drinking in preparation for the first medication session (See Table 2). Among participants who subsequently received psilocybin, PHDD decreased by a mean of 32.37 (95% Cl: 23.68-41.07), Hedges' g=1.08 (95% Cl: 0.74-1.47). Similar changes in PHDD were observed among participants who subsequently received diphenhydramine (mean decrease=27.26 [95% Cl: 20.83-33.69], Hedges' g=1.02 [95% Cl: 0.75-1.44]).

### Efficacy

### Continuous Drinking Outcomes

The primary outcome analysis demonstrated a main effect of treatment on the three-dimensional drinking outcome vector (F_{(1,86)}=6.18, p=0.015). During the weeks 5-36, participants who received psilocybin had lower PHDD than those who received diphenhydramine (9.71 [SD=26.21] vs. 23.57 [SD=26.21], mean difference =13.86 [95% Cl: 3.00-24.72], Hedges' g=0.52, p=0.013). Results for the secondary continuous drinking outcomes, PDD and DPD, are shown in Table 2.

### Dichotomous Drinking Outcomes and Problems Related to Drinking

Psilocybin-treated participants were more likely than those receiving diphenhydramine have no heavy drinking days and to have a 2-level reduction in WHO risk level during weeks 5-36 (Table 3). During the final month of follow-up (weeks 33-36), these differences persisted, and the rates of abstinence as well as 1-and 3-level reductions in WHO risk levels were also higher in the psilocybin-treated participants than in the diphenhydramine group. Numbers needed to treat for these outcomes ranged from 4.0 to 8.2, and odds ratios ranged from 2.03 to 4.00. Psilocybin-treated participants also showed moderate to large reductions in several categories of drinking-related problems at week 24 and/or week 36 (Table 1). Including all available data at the final double-blind timepoint (week 36), the Total Problems score was 6.59 [SD=8.80] in those who received psilocybin vs. 13.00 [SD=10.48] in those who received diphenhydramine (mean difference=6.41 [95% Cl: 2.22-10.60], Hedges' g=0.67, p=0.003).

### Safety

A total of 204 adverse events (119 in the psilocybin group and 85 within the diphenhydramine group) were reported during the 32 weeks following the first administration of study medication (Table 2). Three serious adverse events were reported, all in the diphenhydramine group. One participant had two psychiatric admissions due to suicidal ideation reported during binge drinking episodes. A second participant was hospitalized for a Mallory-Weiss tear due to severe vomiting during a binge drinking episode.

Certain treatment-emergent adverse events occurred within 48 hours of study drug administration. Headaches were common after psilocybin administration, occurring in 21/48 (43.8%) participants who received psilocybin vs. 2/45 (4.4%) who received diphenhydramine. Anxiety and nausea were also reported more frequently during psilocybin administration sessions. Two participants assigned to psilocybin received diazepam 10 mg by mouth for anxiety during their second medication session. The anxiety resolved within 45 and 210 minutes in these cases. One participant assigned to psilocybin reported passive suicidal ideation for 15 minutes during a medication session, which resolved without sequelae. There were no persistent disturbances suggestive of psychosis or hallucinogen persisting perception disorder.

### VI—Discussion

In this randomized controlled trial of psilocybin-assisted treatment of AUD, psilocybin treatment was associated with improved drinking outcomes during 32 weeks of double-blind observation. PHDD among psilocybin-treated participants was 41% of that observed in the diphenhydramine-treated group. Exploratory analyses confirmed the between-group effect across a range of secondary drinking measures.

Adverse events associated with psilocybin administration were mostly mild and self-limiting, consistent with other recent trials evaluating the effects of psilocybin in various conditions.¹⁻⁸ However, it must be emphasized that these safety findings cannot be generalized to other contexts. The study implemented measures to ensure safety including careful medical and psychiatric screening, therapy and monitoring provided by two well-trained therapists including a licensed psychiatrist, and the availability of medications to treat acute psychiatric reactions.

### VII—Conclusions

In this randomized controlled trial in participants with AUD, psilocybin administered in combination with psychotherapy was associated with robust and sustained decreases in drinking which were greater than those observed following active placebo and psychotherapy.

TABLE 1
Participants Characteristics

| | Mean (SD) | | |
|---|---|---|---|
| | Total | Diphenhydramine | Psilocybin |
| No. | 95 | 46 | 49 |

Demographic characteristics

| | | | |
|---|---|---|---|
| Age, y | 45.78 (11.56) | 44.24 (12.15) | 47.18 (10.93) |
| Household income, median | 100 000 (3700- | 110 000 (8000- | 100 000 (3700- |
| (range), $ | 4 000 000) | 800 000) | 4 000 000) |

Sex

| | | | |
|---|---|---|---|
| Female | 42 (44.2) | 21 (45.7) | 21 (42.9) |
| Male | 53 (55.8) | 25 (54.3) | 28 (57.1) |

Race and ethnicity, No. (%)^{a}

| | | | |
|---|---|---|---|
| American Indian/Alaska Native | 1 (1.1) | 1 (2.2) | 0 |
| Black | 5 (5.3) | 1 (2.1) | 4 (8.2) |
| Hispanic | 16 (16.8) | 8 (17.4) | 8 (16.3) |
| Non-Hispanic White | 75 (78.9) | 37 (80.4) | 38 (77.6) |

Drinking-related characteristics

| | | | |
|---|---|---|---|
| % Drinking days | 74.85 (28.06) | 71.00 (29.02) | 78.47 (26.92) |
| % Heavy drinking days | 52.71 (30.58) | 47.93 (28.74) | 57.20 (31.84) |
| Drinks per day | 4.78 (2.62) | 4.33 (2.39) | 5.20 (2.78) |
| Drinks per drinking day | 7.10 (4.05) | 6.64 (3.37) | 7.52 (4.58) |
| No. of dependence criteria^{b} | 5.25 (1.22) | 5.41 (1.20) | 5.10 (1.23) |
| Age at onset, y | 31.42 (11.42) | 30.96 (12.03) | 31.86 (10.92) |
| Years dependent | 14.20 (9.68) | 13.00 (10.31) | 15.33 (9.00) |
| Short Index of Problems | 20.98 (9.15) | 21.60 (9.61) | 20.26 (8.89) |
| (total score) | | | |

WHO risk category, No. (%)^{b}

| | | | |
|---|---|---|---|
| Very high | 30 (31.6) | 12 (26.1) | 18 (36.7) |
| High | 32 (33.7) | 15 (32.6) | 17 (34.7) |
| Moderate | 21 (22.1) | 12 (26.1) | 9 (18.4) |
| Low | 12 (12.6) | 7 (15.2) | 5 (10.2) |

Abbreviation: WHO, World Health Organization.
^{a}Race and ethnicity were determined by participant self-report according to standard National Institutes of Health categories in order to assess the representativeness of the sample. Sum is greater than 100% due to multiple categories selected by 2 participants.
^{b}Defined using the Structured Clinical Interview for DSM-IV axis I disorders.⁴⁰
^{c} WHO risk categories are defined as follows. Abstinence was defined as no risk (level 0), following a recent study evaluating the use of WHO risk levels as a treatment outcome.⁵³ For men, low risk (level 1) is defined as >0 g/d to ≤40 g/d; moderate risk (level 2) as >40 g/d to ≤60 g/d; high risk (level 3) as >60 g/d to ≤100 g/d; and very high risk (level 4) as >100 g/d. For women, low risk (level 1) is defined as >0 g/d to ≤20 g/d; moderate risk (level 2) as >20 g/d to ≤40 g/d; high risk (level 3) as >40 g/d to ≤60 g/d; and very high risk (level 4) as >60 g/d. Change in WHO risk level was calculated in relation to drinking during the 12 weeks prior to screening.

Table 1 presents demographic, alcohol-related, and psychiatric characteristics of the randomized sample. Race and ethnicity were determined by participant self-report in order to assess the representativeness of the sample.

TABLE 2
Between-and Within-Group Treatment Effects^{a}

| Mean (SD) | | | | | |
|---|---|---|---|---|---|
| Diphenhydramine | Psilocybin | | Mean difference | Hedges g | ρ |
| (n = 45) | (n = 48) | Effect | (95% CI) | (95% CI) | value^{b} |

% of Heavy drinking days

| | | | | | | | |
|---|---|---|---|---|---|---|---|
| Screening | 48.57 | 56.48 | Within- | Diphenhydramine | 27.26 | 1.02 | <.001 |
| | (28.73) | (31.77) | group | | (20.83-33.69) | (0.75-1.44) | |
| Week 4^{c} | 21.31 | 24.11 | screening, | Psilocybin | 33.37 | 1.08 | <.001 |
| | (20.14) | (26.29) | week 4 | | (23.68-41.07) | (0.74-1.47) | |
| Follow- | 23.57 | 9.71 | Between- | Diphenhydramine- | 13.86 | 0.52 | .01 |
| up^{d} | (26.67) | (26.21) | group | psilocybin | (3.00-24.72) | (0.11-0.94) | |
| | | | follow-up | | | | |

% of Drinking days

| | | | | | | | |
|---|---|---|---|---|---|---|---|
| Screening | 71.68 | 78.03 | Within- | Diphenhydramine | 25.68 | 0.85 | <.001 |
| | (28.98) | (27.02) | group | | (19.19-32.18) | (0.58-1.14) | |
| Week 4^{c} | 45.99 | 52.98 | screening, | Psilocybin | 25.05 | 0.83 | <.001 |
| | (30.40) | (31.78) | week 4 | | (16.92-33.18) | (0.53-1.16) | |
| Follow- | 42.83 | 29.39 | Between- | Diphenhydramine- | 13.44 | 0.4 | .05 |
| up^{d} | (33.43) | (32.86) | group | psilocybin | (−0.18 to 27.05) | (−0.01 to 0.82) | |
| | | | follow-up | | | | |

Drinks per day

| | | | | | | | |
|---|---|---|---|---|---|---|---|
| Screening | 4.38 | 5.2 | Within- | Diphenhydramine | 2.19 | 0.97 | <.001 |
| | (2.39) | (2.81) | group | | (1.65-2.73) | (0.68-1.31) | |
| Week 4^{c} | 2.19 | 2.77 | screening, | Psilocybin | 2.43 | 0.91 | <.001 |
| | (1.98) | (2.30) | week 4 | | (1.87-3.00) | (0.66-1.23) | |
| Follow- | 2.26 | 1.17 | Between- | Diphenhydramine- | 1.09 | 0.54 | .01 |
| up^{d} | (2.02) | (1.99) | group | psilocybin | (0.27-1.92) | (0.13-0.96) | |
| | | | follow-up | | | | |

^{a}Positive between-group effect sizes signify lower (more favorable) means in the psilocybin group. Positive within-group effect sized signify improvement between screening and week 4.
^{b}ρ values for within-group comparisons are based on paired t test with no correction for multiple comparisons. ρ values for between-group comparisons represent univariate marginal between-group contrasts from the primary outcome analysis (multivariate analysis of variance).
^{c}Represents the 4 weeks prior to administration of study medication.
^{d}Represents the 32-week double-blind follow-up period.

In Table 2, means and standard deviations are shown for each treatment group (Diphenhydramine: n=45; Psilocybin: n=48) at Screening, Week 4 (representing the 4 weeks prior to administration of study medication), and the 32-week double-blind follow-up period. Mean differences within and between groups are shown with 95% confidence intervals, and Hedge's g is provided with 95% confidence intervals as a measure of effect size.

TABLE 3
Treatment Effects on Dichotomous Drinking Outcomes

| | | No. (%)^{a} | | | | | | |
|---|---|---|---|---|---|---|---|---|
| | Follow-up | Diphenhydramine | | Psilocybin | | | | ρ |
| | period | (n = 45) | | (n = 48) | NNT | OR (95% CI)^{b} | | value^{b, c} |
| Abstinence | Weeks 5-36 | 4 | (8.9) | 11 (22.9) | 7.1 | 3.05 | (0.89-10.40) | .06 |
| | Weeks 33-36 | 11 | (24.4) | 23 (47.9) | 4.3 | 2.84 | (1.17-6.89) | .02 |
| No heavy drinking | Weeks 5-36 | 5 | (11.1) | 16 (33.3) | 4.5 | 4 | (1.32-12.10) | .01 |
| | Weeks 33-36 | 18 | (40.0) | 30 (62.5) | 4.4 | 2.5 | (1.08-5.76) | .03 |

WHO risk level^{d}

| | | | | | | | | |
|---|---|---|---|---|---|---|---|---|
| Decrease 1 | Weeks 5-36 | 32 | (71.1) | 40 (83.3) | 8.2 | 2.03 | (0.75-5.50) | .16 |
| | Weeks 33-36 | 29 | (64.4) | 43 (89.6) | 4 | 4.74 | (1.57-14.39) | .004 |
| Decrease 2 | Weeks 5-36 | 18 | (40.0) | 29 (60.4) | 4.9 | 2.29 | (1.00-5.26) | .049 |
| | Weeks 33-36 | 18 | (40.0) | 29 (60.4) | 4.9 | 2.29 | (1.00-5.26) | .049 |
| Decrease 3 | Weeks 5-36 | 6 | (13.3) | 14 (29.2) | 6.3 | 2.68 | (0.93-7.73) | .06 |
| | Weeks 33-36 | 8 | (17.8) | 18 (37.5) | 5.1 | 2.78 | (1.06-7.26) | .03 |

Abbreviation: NNT, number needed to treat; OR odds ratio; WHO, World Health Organization.
^{a}Number and proportion of participants within each treatment group that met dichotomous drinking outcomes for the 32-week double-blind follow-up period following the first medication administration session (weeks 5-36) and the final 4 weeks of double-blind observation (weeks 33-36).
^{b}Confidence intervals and ρ values have not been corrected for multiple comparisons.
^{c}Nominal ρ value, Pearson χ².
^{d}WHO risk levels are defined as follows. Abstinence was defined as no risk (level 0), following a recent study evaluating the use of WHO risk levels as a treatment outcome.⁵³ For men, low risk (level 1) is defined as >0 g/d to ≤40 g/d; moderate risk (level 2) as >40 g/d to ≤60 g/d; high risk (level 3) as >60 g/d to ≤100 g/d; and very high risk (level 4) as >100 g/d. For women, low risk (level 1) is defined as >0 g/d to ≤20 g/d; moderate risk (level 2) as >20 g/d to ≤40 g/d; high risk (level 3) as >40 g/d to ≤60 g/d; and very high risk (level 4) as >60 g/d. Change in WHO risk level was calculated in relation to drinking during the 12 weeks prior to screening.

### REFERENCES

1. Griffiths R R, Johnson M W, Carducci M A, et al. Psilocybin produces substantial and sustained decreases in depression and anxiety in patients with life-threatening cancer: A randomized double-blind trial. Journal of Psychopharmacology. 2016;30(12):1181-1197.
2. Ross S, Bossis A, Guss J, et al. Rapid and Sustained Symptom Reduction Following Psilocybin Treatment for Anxiety and Depression in Patients with Life-Threatening Cancer: A Randomized Controlled Trial. J Psychopharmacol. 2016;30(12):1165-1180.
3. Bogenschutz M P, Forcehimes A A, Pommy J A, Wilcox C E, Barbosa P C, Strassman R J. Psilocybin-assisted treatment for alcohol dependence: a proof-of-concept study. J Psychopharmacol. 2015;29(3):289-299.
4. Johnson M W, Garcia-Romeu A, Cosimano M P, Griffiths R R. Pilot study of the 5-HT2AR agonist psilocybin in the treatment of tobacco addiction. J Psychopharmacol. 2014;28(11):983-992.
5. Carhart-Harris R, Giribaldi B, Watts R, et al. Trial of Psilocybin versus Escitalopram for Depression. N Engl J Med. 2021;384(15):1402-1411.
6. Davis A K, Barrett F S, May D G, et al. Effects of Psilocybin-Assisted Therapy on Major Depressive Disorder: A Randomized Clinical Trial. JAMA Psychiatry. 2021;78(5):481-489.
7. Carhart-Harris R L, Bolstridge M, Rucker J, et al. Psilocybin with psychological support for treatment-resistant depression: an open-label feasibility study. The lancet Psychiatry. 2016;3(7):619-627.
8. Grob C S, Danforth A L, Chopra G S, et al. Pilot study of psilocybin treatment for anxiety in patients with advanced-stage cancer. Arch Gen Psychiatry. 2011;68(1):71-78.
9. Daws R E, Timmermann C, Giribaldi B, et al. Increased global integration in the brain after psilocybin therapy for depression. Nature medicine. 2022;28(4):844-851.
10. Halberstadt A L. Recent advances in the neuropsychopharmacology of serotonergic hallucinogens. Behav Brain Res. 2015;277:99-120.
11. Nichols D E. Hallucinogens. Pharmacol Ther. 2004;101(2):131-181.
12.Aleksandrova L R, Phillips A G. Neuroplasticity as a convergent mechanism of ketamine and classical psychedelics. Trends in pharmacological sciences. 2021;42(11):929-942.
13. Doss M K, Povazan M, Rosenberg M D, et al. Psilocybin therapy increases cognitive and neural flexibility in patients with major depressive disorder. Translational psychiatry. 2021;11(1):574.
14. Carhart-Harris R L, Friston K J. REBUS and the Anarchic Brain: Toward a Unified Model of the Brain Action of Psychedelics. Pharmacological reviews. 2019;71(3):316-344.
15. Catlow B J, Song S, Paredes D A, Kirstein C L, Sanchez-Ramos J. Effects of psilocybin on hippocampal neurogenesis and extinction of trace fear conditioning. Exp Brain Res. 2013;228(4):481-491.
16. Kelly J R, Gillan C M, Prenderville J, et al. Psychedelic Therapy's Transdiagnostic Effects: A Research Domain Criteria (RDoC) Perspective. Front Psychiatry. 2021;12:800072.
17. Roseman L, Demetriou L, Wall M B, Nutt D J, Carhart-Harris R L. Increased amygdala responses to emotional faces after psilocybin for treatment-resistant depression. Neuropharmacology. 2018;142:263-269.
18. Meinhardt M W, Pfarr S, Fouquet G, et al. Psilocybin targets a common molecular mechanism for cognitive impairment and increased craving in alcoholism. Sci Adv. 2021;7(47):eabh2399.
19. Mertens L J, Wall M B, Roseman L, Demetriou L, Nutt D J, Carhart-Harris R L. Therapeutic mechanisms of psilocybin: Changes in amygdala and prefrontal functional connectivity during emotional processing after psilocybin for treatment-resistant depression. J Psychopharmacol. 2020;34(2):167-180.
20. Mertens L J, Preller K H. Classical Psychedelics as Therapeutics in Psychiatry-Current Clinical Evidence and Potential Therapeutic Mechanisms in Substance Use and Mood Disorders. Pharmacopsychiatry. 2021;54(4):176-190.
21. Barrett F S, Doss M K, Sepeda N D, Pekar J J, Griffiths R R. Emotions and brain function are altered up to one month after a single high dose of psilocybin. Sci Rep. 2020;10(1):2214.
22. Goldberg S B, Shechet B, Nicholas C R, et al. Post-acute psychological effects of classical serotonergic psychedelics: a systematic review and meta-analysis. Psychol Med. 2020;50(16):2655-2666.
23. Cameron L P, Benson C J, Dunlap L E, Olson D E. Effects of N, N-Dimethyltryptamine on Rat Behaviors Relevant to Anxiety and Depression. ACS chemical neuroscience. 2018;9(7):1582-1590.
24. Shao L X, Liao C, Gregg I, et al. Psilocybin induces rapid and persistent growth of dendritic spines in frontal cortex in vivo. Neuron. 2021;109(16):2535-2544 e2534.
25. Hesselgrave N, Troppoli T A, Wulff A B, Cole A B, Thompson S M. Harnessing psilocybin: antidepressant-like behavioral and synaptic actions of psilocybin are independent of 5-HT2R activation in mice. Proceedings of the National Academy of Sciences of the United States of America. 2021;118(17).
26. Roseman L, Nutt D J, Carhart-Harris R L. Quality of Acute Psychedelic Experience Predicts Therapeutic Efficacy of Psilocybin for Treatment-Resistant Depression. Frontiers in pharmacology. 2017;8:974.
27. Davis A K, Barrett F S, Griffiths R R. Psychological flexibility mediates the relations between acute psychedelic effects and subjective decreases in depression and anxiety. J Contextual Behav Sci. 2020;15:39-45.
28. Garcia-Romeu A, Griffiths R R, Johnson M W. Psilocybin-occasioned mystical experiences in the treatment of tobacco addiction. Curr Drug Abuse Rev. 2014;7(3):157-164.
29.Yaden D B, Griffiths R R. The Subjective Effects of Psychedelics Are Necessary for Their Enduring Therapeutic Effects. ACS Pharmacol Transl Sci. 2021;4(2):568-572.
30. Rieser N M, Herdener M, Preller K H. Psychedelic-Assisted Therapy for Substance Use Disorders and Potential Mechanisms of Action. Current topics in behavioral neurosciences. 2021.
31. Murphy R, Kettner H, Zeifman R, et al. Therapeutic Alliance and Rapport Modulate Responses to Psilocybin Assisted Therapy for Depression. Frontiers in pharmacology. 2021;12:788155.
32. Greenway K T, Garel N, Jerome L, Feduccia A A. Integrating psychotherapy and psychopharmacology: psychedelic-assisted psychotherapy and other combined treatments. Expert Rev Clin Pharmacol. 2020;13(6):655-670.
33. Smart R G, Storm T, Baker EF, Solursh L. A controlled study of lysergide in the treatment of alcoholism. 1. The effects on drinking behavior. Q J Stud Alcohol. 1966;27(3):469-482.
34. Hollister LE , Shelton J, Krieger G. A controlled comparison of lysergic acid diethylamide (LSD) and dextroamphetamine in alcoholics.Am J Psychiatry. 1969;125(10):1352-1357.
35. Ludwig A, Levine J, Stark L, Lazar R. A clinical study of LSD treatment in alcoholism. Am J Psychiatry. 1969;126(1):59-69.
36. Bowen W T, Soskin R A, Chotlos J W. Lysergic acid diethylamide as a variable in the hospital treatment of alcoholism: a follow-up study. J Nery Ment Dis. 1970;150(2):111-118.
37. Pahnke W N, Kurland A A, Unger S, Savage C, Grof S. The experimental use of psychedelic (LSD) psychotherapy. JAMA. 1970;212(11):1856-1863.
38. Tomsovic M, Edwards R V. Lysergide treatment of schizophrenic and nonschizophrenic alcoholics: a controlled evaluation. Q J Stud Alcohol. 1970;31(4):932-949.
39. Krebs T S, Johansen P O. Lysergic acid diethylamide (LSD) for alcoholism: meta-analysis of randomized controlled trials. J Psychopharmacol. 2012;26(7):994-1002.
40. First M B, Spitzer R L, Gibbon M, Williams J B W. Structured Clinical Interview for DSM-IV Axis I Disorders-Patient Edition. New York, NY: Biometrics Research Department, New York State Psychiatric Institute; 1997.
41. Bogenschutz M P, Forcehimes A A. Development of a Psychotherapeutic Model for Psilocybin-assisted Treatment of Alcoholism. Journal of Humanistic Psychology. 2017;57(4):389-414.
42. Griffiths R R, Richards W A, McCann U, Jesse R. Psilocybin can occasion mystical-type experiences having substantial and sustained personal meaning and spiritual significance. Psychopharmacology (Berl). 2006;187(3):268-283; discussion 284-292.
43. Sobell L C, Sobell M B. Timeline follow-back: A technique for assessing self-reported alcohol consumption. In: Litten RA, Allen JP, eds. Measuring alcohol consumption: Psychosocial and biological methods. Totowa, NJ: Humana Press; 1992.
44. Sobell L C, Sobell M B. Timeline Follow Back: A calendar method for assessing alcohol and drug use (User's Guide). Toronto: Addiction Research Foundation; 1996.
45. Center for Drug Evaluation and Research. Alcoholism: Developing Drugs for Treatment: Guidance for Industry. Silver Spring, MD: US Food and Drug Administration, Center for Drug Evaluation and Research;2015.
46. Sobell L C, Sobell M B, Leo G I, Cancilla A. Reliability of a timeline method: assessing normal drinkers' reports of recent drinking and a comparative evaluation across several populations. British journal of addiction. 1988; 83:393-402.
47. Sobell L C, Brown J, Leo G I, Sobell M B. The reliability of the Alcohol Timeline Followback when administered by telephone and by computer. Drug and Alcohol Dependence. 1996;42(1):49-54.
48. World Health Organization. International Guide for Monitoring Alcohol Consumption and Related Harm. Geneva, Switzerland: World Health Organization; 2000.
49. Miller W R, Tonigan J S, Longabaugh R. The Drinker Inventory of Consequences (DrInC): An instrument for assessing adverse consequences of alcohol abuse. Test Manual (Vol. 4). Rockville, MD: US Government Printing Office; 1995.
50. Gamble C, Krishan A, Stocken D, et al. Guidelines for the Content of Statistical Analysis Plans in Clinical Trials. JAMA. 2017;318(23):2337-2343.
51.van Buuren S, Groothuis-Oudshoorn K. mice: Multivariate Imputation by Chained Equations in R. Journal of Statistical Software. 2011;45(3):1-67.
52. Stevens J. Applied multivariate statistics for the social sciences. Mahwah, N.J.: Lawrence Erlbaum Associates; 2002.

1. A method for treating a human subject afflicted with alcohol use disorder comprising administering to the subject an amount of a psilocybin-based compound sufficient to continuously treat the subject for at least 32 weeks, wherein (i) the psilocybin-based compound is administered in two doses four weeks apart, and (ii) each dose is less than 25 mg/70 kg.

2. The method of claim 1, wherein the psilocybin-based compound is psilocybin.

3. The method of claim 1, wherein the administering is oral.

4. The method of claim 1, wherein the subject experiences a reduction in the percent of heavy drinking days for a period of at least 32 weeks following administration of the first dose.

5. The method of claim 4, wherein the subject experiences a reduction in the percent of heavy drinking days for a period of at least one year following administration of the first dose.

6. The method of claim 1, wherein the subject experiences a reduction in the percent of drinking days for a period of at least 32 weeks following administration of the first dose.

7. The method of claim 6, wherein the subject experiences a reduction in the percent of drinking days for a period of at least one year following administration of the first dose.

8. The method of claim 1, wherein the subject experiences a reduction in drinks per day for a period of at least 32 weeks following administration of the first dose.

9. The method of claim 8, wherein the subject experiences a reduction in drinks per day for a period of at least one year following administration of the first dose.

10. A method for treating a human subject afflicted with alcohol use disorder comprising administering to the subject an amount of a psilocybin-based compound sufficient to continuously treat the subject for at least 32 weeks, wherein (i) the psilocybin-based compound is administered in two doses four weeks apart, and (ii) the method further comprises administering to the subject an effective amount of an anti-anxiety drug concurrently with each administration of psilocybin-based compound.

11. The method of claim 10, wherein the psilocybin-based compound is psilocybin.

12. The method of claim 10, wherein the administering is oral.

13. The method of claim 10, wherein the anti-anxiety drug is diazepam.

14. A method for treating a human subject afflicted with alcohol use disorder comprising administering to the subject a single dose of a psilocybin-based compound in an amount sufficient to continuously treat the subject for at least 32 weeks.

15. The method of claim 14, wherein the psilocybin-based compound is psilocybin.

16. The method of claim 14, wherein the administering is oral.

17. The method of claim 14, wherein the dose is from 25 mg/70 kg to 40 mg/70 kg.

18. The method of claim 14 any of claims 14 16, wherein the dose is less than 25 mg/70 kg.

19. The method of claim 14, wherein the subject experiences a reduction in the percent of heavy drinking days for a period of at least 32 weeks following administration.

20. The method of claim 19, wherein the subject experiences a reduction in the percent of heavy drinking days for a period of at least one year following administration.

21. The method of claim 14, wherein the subject experiences a reduction in the percent of drinking days for a period of at least 32 weeks following administration.

22. The method of claim 21, wherein the subject experiences a reduction in the percent of drinking days for a period of at least one year following administration.

23. The method of claim 14, wherein the subject experiences a reduction in drinks per day for a period of at least 32 weeks following administration.

24. The method of claim 23, wherein the subject experiences a reduction in drinks per day for a period of at least one year following administration.

25. The method of claim 14, wherein the method further comprises administering to the subject an effective amount of an anti-anxiety drug concurrently with the administration of psilocybin-based compound.

26. The method of claim 25, wherein the anti-anxiety drug is diazepam.

27. The method of claim 1, wherein the subject is undergoing psychotherapy.
